(19) United States
(12) Patent Application Publication
(10) Pub. No.: US 2011/0190784 A1
(43) Pub. Date: Aug. 4, 2011

(54) CATHETER WITH REMOVABLE SHAPING SKELETON AND METHODS OF USING AND MAKING SAME

(75) Inventors: John Hastings, Savage, MN (US); Mainak Saha, Edina, MN (US)

(73) Assignee: PACESETTER, INC., Sylmar, CA (US)

(21) Appl. No.: 12/700,554

(22) Filed: Feb. 4, 2010

(51) Int. Cl. A61B 17/00

(52) U.S. Cl. 606/129

(57) ABSTRACT

A delivery tool for the delivery of an implantable medical lead includes a longitudinally extending tubular body and a longitudinally extending skeleton. The longitudinally extending tubular body includes a distal end, a proximal end, a tubular body segment proximal the distal end, a first lumen extending between the proximal and distal ends, and a second lumen. The first lumen is configured to receive therein the implantable medical lead. The longitudinally extending skeleton is received in the second lumen and includes a distal end, a proximal end, and a portion near the distal end that biases into a non-linear shape. The portion of the skeleton causes the tubular body segment to generally assume the non-linear shape. The skeleton is withdrawable from the second lumen. The proximal ends of the skeleton and the tubular body may have a mechanical engagement arrangement that mechanically engages the proximal ends together in a manner that may be released to allow the proximal ends to be separated from each other. Upon withdrawal of the skeleton from the second lumen, the tubular body segment is free to assume a shape different from the non-linear shape.

### FIELD OF THE INVENTION

The present invention relates to medical apparatus and methods. More specifically, the present invention relates to delivery tools used in the implantation of implantable medical leads. The present invention also relates to methods of manufacturing and using such delivery tools.

### BACKGROUND OF THE INVENTION

To provide electrotherapy (e.g., cardiac resynchronization therapy (CRT)) to a patient's heart, medical leads are implanted in the patient's chest to extend from an implantable pulse generator (e.g., an implantable cardioverter defibrillator (ICD) or pacemaker), also implanted in the patient's chest, to a therapy location in or on the patient's heart. Delivery catheters are used to place the lead distal end at the desired therapy location. Specifically, these catheters navigate the venous system and cardiac anatomy to locate critical anatomical locations and serve as a conduit through which leads are delivered. Once the lead distal end is located at the therapy location in or on the patient's heart, the catheter must be removed from about the lead without disrupting the lead distal end from the therapy location.

Two primary catheter types have been developed to accommodate the removal process. The first type is referred to as a “slittable” catheter, which is designed to be sliced over the body of the lead using a cutting tool known as a slitter. The second type of delivery catheter is referred to as a “peelable” catheter, which is designed to be split by hand at the hub of the catheter and then peeled into two halves over the body of the lead.

Although current slittable and peelable catheter types seek to minimize lead movement or dislodgement upon catheter removal from about an implanted lead, residual risk is still high. In fact, many physicians feel catheter removal is the highest risk and most frustrating process of the entire lead implantation process. Physicians may spend an hour implanting a lead, only to have the lead dislodge upon catheter removal.

A primary contributor to lead dislodgement is “catheter whipping”, which can be understood from the following discussion regarding FIGS. 1-3. As shown in FIG. 1, which is a diagrammatic depiction of a delivery catheter 5 being navigated through the vasculature and cardiac anatomy to a desired implantation site 7, the delivery catheter 5 includes a proximal end 10, a distal end 15, and a tubular body 20 extending between the proximal 10 and distal ends 15. The distal end 15 of the catheter 5 is formed into a specific curved geometry to accommodate, for example, cannulation of the coronary sinus 25 and sub branch thereof or to gain access to other cardiac anatomy as the specific lead implantation may require. For example, the catheter 5 having a distal end 15 self-biased into a curve 30 may be navigated through the subclavian vein 35, the superior vena cava 40 and into the right atrium 45 of the heart 50. The configuration of the curve 50 assists the physician in navigating the distal end 15 into the coronary sinus 25 and a sub-branch thereof via the ostium (“OS”) 55.

The curve 30 may be heat set or otherwise formed into the distal end 15 such that the curve 30 may be straightened as needed via application of a straightening force to the curve 30 during navigation of the distal end 15 through the vasculature and cardiac anatomy, but will spring back into its self-biased curved shape upon removal of the straightening force from the curve 30. Thus, as can be understood from FIG. 2, which is the same diagrammatic depiction as FIG. 1, except the catheter distal end 15 has now been navigated into the coronary sinus 25 and a sub branch thereof, the walls of the coronary sinus 25 and the sub branch thereof exert a straightening force on the distal end 15 such that the curve 30 is straightened as the distal end 15 is advanced along the coronary sinus 25 and the sub branch thereof to deliver a distal end 60 of an implantable medical lead 65 to the desired lead implantation site 7. As indicated in FIG. 2, such an implantable medical lead 65 will have a lead connector end 67 at the lead proximal end 70 for connecting to a pulse generator 75, as indicated by arrow A, once the lead distal end 60 is successfully delivered to the implantation site 7 and the catheter 5 is withdrawn from about the implanted lead 65 without dislodging the lead distal end 60 from the implantation site 7.

As illustrated in FIG. 3, which is the same diagrammatic depiction as FIG. 2, except the catheter 5 is in the process of being withdrawn from about the lead 60, once the catheter distal end 15 is free of the restraint of the walls of the coronary sinus 25 and the sub branch thereof and the associated straightening force, the curve 30 self-biases back into its natural curved shape, causing the catheter distal end 15 to whip away from the OS 55, as indicated by arrow B. The whipping of the catheter distal end 15 jerks the implanted lead 65 such that the lead distal end 60 is dislodged away from the desired lead implantation site 7, as indicated by arrow C. The lead implantation process must then begin anew, increasing procedure time, risk and cost.

There is a need in the art for a delivery catheter configured to reduce, if not eliminate, the possibility of catheter whip. There is also a need in the art for a method of manufacturing such a lead and a method of lead implantation that reduces the likelihood of lead dislodgement from a desired implantation site.

### BRIEF SUMMARY OF THE INVENTION

A delivery tool for the delivery of an implantable medical lead is disclosed herein. In one embodiment, the tool includes a longitudinally extending tubular body and a longitudinally extending structural member (i.e., skeleton). The longitudinally extending tubular body includes a distal end, a proximal end, a tubular body segment proximal the distal end, a first lumen extending between the proximal and distal ends, and a second lumen. The first lumen is configured to receive therein the implantable medical lead. The longitudinally extending skeleton is received in the second lumen and includes a distal end, a proximal end, and a portion near the distal end that biases into a non-linear shape. The portion of the skeleton causes the tubular body segment to generally assume the non-linear shape. The skeleton is withdrawable from the second lumen. The proximal ends of the skeleton and the tubular body may have a mechanical engagement arrangement that mechanically engages the proximal ends together in a manner that may be released to allow the proximal ends to be separated from each other. Upon withdrawal of the skeleton from the second lumen, the tubular body segment is free to assume a shape different from the non-linear shape.

A method of delivering a distal end of an implantable medical lead to a target implant site in a patient is also disclosed herein. In one embodiment the method includes: a) providing a delivery tool in an assembled state, wherein the delivery tool in the assembled state includes: a longitudinally extending tubular body including a distal end, a proximal end, a tubular body segment proximal the distal end, a first lumen extending between the proximal and distal ends, and a second lumen; and a longitudinally extending skeleton received in the second lumen and including a distal end, a proximal end, and a portion near the distal end that biases into a non-linear shape, wherein the portion of the skeleton causes the tubular body segment to generally assume the non-linear shape; b) tracking the delivery tool in the assembled state through a route in the patient; c) distally displacing the implantable medical lead through the first lumen of the delivery tool in the assembled state until the a distal end of the implantable medical lead is located as desired near the target implant site; d) upon achieving step c), transitioning the delivery tool from the assembled state to a disassembled state, wherein the delivery tool in the disassembled state has the skeleton removed from the second lumen; and e) upon achieving step d), proximally withdrawing the distal end of the tubular body from the distal end of the implantable medical lead.

While multiple embodiments are disclosed, still other embodiments of the present invention will become apparent to those skilled in the art from the following Detailed Description, which shows and describes illustrative embodiments of the invention. As will be realized, the invention is capable of modifications in various aspects, all without departing from the spirit and scope of the present invention. Accordingly, the drawings and detailed description are to be regarded as illustrative in nature and not restrictive.

### BRIEF DESCRIPTION OF THE DRAWINGS

FIG. 1 is a diagrammatic depiction of a delivery catheter being navigated through the vasculature and cardiac anatomy to a desired implantation site, the catheter distal end being self-biased in a curved configured to facilitate access of the catheter distal end into the coronary sinus and sub branch thereof.

FIG. 2 is the same diagrammatic depiction as FIG. 1, except the catheter distal end has now been navigated into the coronary sinus and sub branch thereof to deliver a lead to the desired implantation site, the walls of the coronary sinus and sub branch thereof exerting a straightening force on the catheter distal end such that the curve is straightened from its self-biased configuration.

FIG. 3 is the same diagrammatic depiction as FIG. 2, except the catheter is in the process of being withdrawn from about the lead and is free of the restraint of the walls of the coronary sinus and sub branch thereof, the catheter distal end biasing back into the self-biased curved configuration and dislodging the lead distal end from the desired implantation site.

FIG. 4 is a diagrammatic depiction of the catheter in an assembled state and in longitudinal cross section, a lead to be delivered through the catheter, and a pulse generator for connection to the lead distal end.

FIG. 5 is a transverse cross section of the catheter in an assembled state and as taken along section line 5-5 in FIG. 4.

FIG. 6 is the same view of the catheter depicted in FIG. 4, except the catheter is in a disassembled state.

FIG. 7 is a flow chart outlining a method of employing the catheter.

FIG. 8 is an isometric view of the proximal ends of the skeleton and tubular body with a lead extending into the catheter, wherein the skeleton includes a U-shaped hub.

FIG. 9 is an isometric view of the proximal ends of the skeleton and tubular body with a lead extending into the catheter, wherein the skeleton includes a splittable hub.

### DETAILED DESCRIPTION

A delivery tool (i.e., catheter or sheath) 100 for the delivery of an implantable medical lead 65 is disclosed herein. In one embodiment, the delivery catheter 100 includes a tubular body 105 and a skeleton 110 received in and coupled to the tubular body 105 when the catheter 100 is in an assembled state. The catheter 100 is employed in the assembled state to deliver the distal end 60 of an implantable medical lead 65 to a target implant site. The catheter 100 may then be transitioned into a disassembled state by decoupling and removing the skeleton 110 from the tubular body 105 as the tubular body 105 remains in place in the patient. Once in the disassembled state, the disassembled catheter 100, or more specifically, the remaining tubular body 105 is removed from the patient and about the implanted lead 65. Thus, the catheter 100 offers the benefit of having a shaped end for tracking through patient vasculature and cardiac anatomy, but may be removed in stages so as to prevent catheter whip and lead dislodgement.

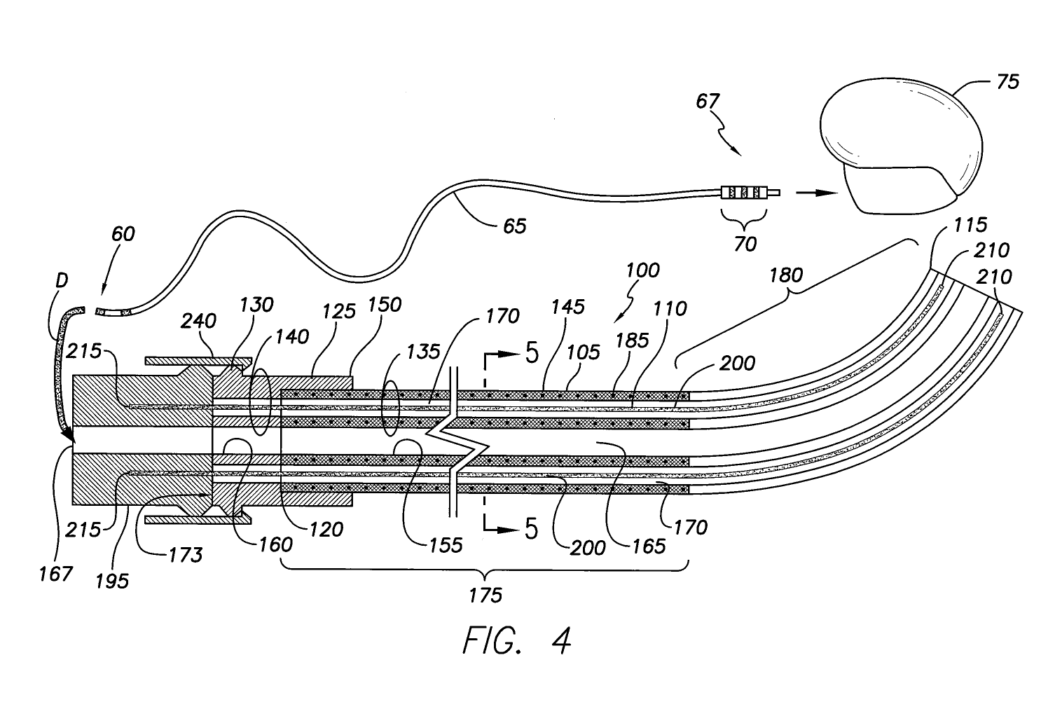

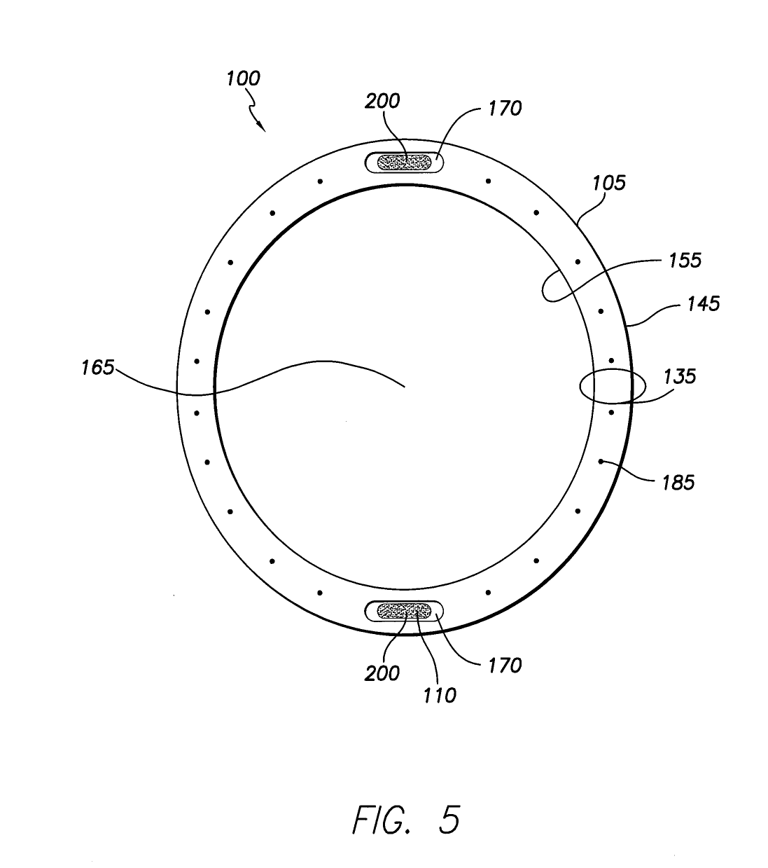

To begin a detailed discussion of the catheter 100, reference is made to FIGS. 4 and 5. FIG. 4 is a diagrammatic depiction of the catheter 100 in an assembled state and in longitudinal cross section, a lead 65 to be delivered through the catheter 100, and a pulse generator 75 for connection to the lead distal end 67. FIG. 5 is a transverse cross section of the catheter 100 in an assembled state and as taken along section line 5-5 in FIG. 4. As indicated in FIGS. 4 and 5, the catheter 100 includes a longitudinally extending tubular body 105 and a longitudinally extending structural member (i.e., skeleton) 110 extending through the tubular body 105 when the catheter 100 is in an assembled state.

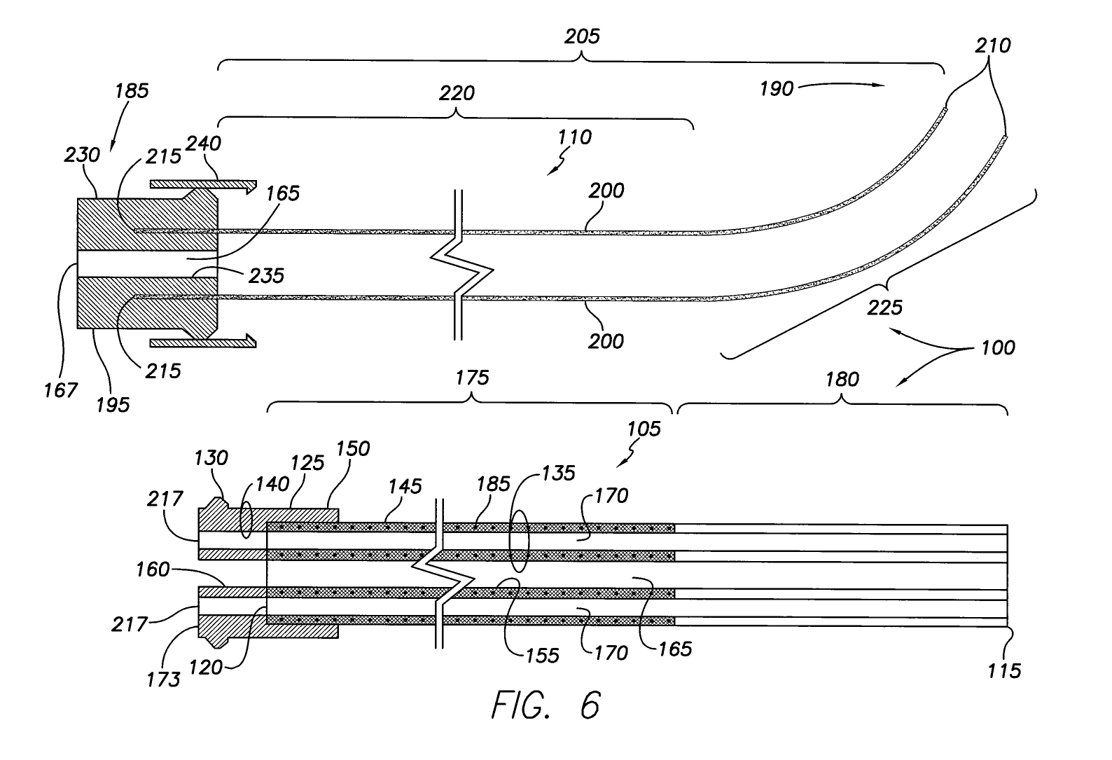

As can be understood from FIG. 6, which is the same view of the catheter 100 depicted in FIG. 4, except the catheter 100 is in a disassembled state, the skeleton 110 may be removed from within the tubular body 105. A comparison of FIGS. 4 and 5 to FIG. 6 illustrates the tubular body 105 of the catheter 100 assumes a curved configuration when the skeleton 110 is received in the tubular body 110 (i.e., the catheter 100 is in the assembled state depicted in FIGS. 4 and 5), and the tubular body 105 is free to assume its natural state (e.g., a straight or even highly flexible or floppy condition) when the skeleton 110 is removed from the tubular body 105 (i.e., the catheter 100 is in the disassembled state depicted in FIG. 6).

As shown in FIG. 6, the longitudinally extending tubular body 105 includes a distal end 115 and a proximal end 120. A hub 125 may be fixedly mounted on the proximal end 120 of the tubular body 105 and may include a first mechanical engagement feature 130. As can be understood from FIGS. 4-6, the tubular body 105 and hub 125 of the tubular body 105 may have respective walls 135, 140 that define the respective outer circumferential surfaces 145, 150 of the tubular body 105 and its hub 125. The respective walls 135, 140 also define respective inner circumferential surfaces 155, 160 that define a central or lead delivery lumen 165 of the tubular body 105 and its hub 125. As indicated by arrow D in FIG. 4, the lead distal end 60 may be fed into the catheter via the proximal opening 167 of the central lumen 165.

As shown in FIGS. 4-6, one, two or more intra-wall lumens 170 extend longitudinally through the tubular body 105 and its hub 125 between the tubular body distal end 115 and the proximal end 173 of the tubular body's hub 125. The intra-wall lumens 170 are defined in the material forming the walls 135, 140 between the respective inner circumferential surfaces 155, 160 and the respective outer circumferential surfaces 145, 150.

In one embodiment, the intra-wall lumens 170 are in a spaced-apart arrangement wherein the intra-wall lumens are evenly radially spaced-apart from each other. For example, in one embodiment, two intra-wall lumens 170 are on opposite sides of the tubular body 105 (i.e., spaced radially apart by 180 degrees).

The hub 125 of the tubular body 105 may be formed of a polymer material such as, for example, polyether block amide (“PEBAX®”), Acrylonitrile-Butadiene-Styrene copolymer (“ABS”), Nylon, Polyethylene, or etc. The proximal end 120 of the tubular body 105 may be received within the hub 125 of the tubular body 105 and fixedly secured thereto via an adhesive, chemical or laser welding, co-molding or extrusion processes, heat forming, or other methods known in the art.

In one embodiment, as illustrated in FIGS. 4 and 6, the tubular body 105 may include a proximal segment 175 and a distal segment 180. The proximal segment may 175 may form the majority of the length of the tubular body 105, the distal segment 180 forming the remainder of the length of the tubular body 105. The proximal segment 175 and distal segment 180 may have different constructions and/or different stiffness and/or torqueability. In one embodiment, for example, the proximal segment 175 may be reinforced with one or more braid layers 185, and the distal segment 180 may be free of such reinforcement. As a result and absent the skeleton 110 when the catheter 100 is in the disassembled state depicted in FIG. 6, the proximal segment 175 may be substantially more stiff and/or torqueable than the distal segment 180. The proximal segment 175 may be formed of a polymer material such as, for example, PEBAX®, High-density polyethylene (“HDPE”), Polyurethane, or etc., and the distal segment 180 may be formed of a polymer material such as, for example, PEBAX®, HDPE, Polyurethane, or etc.

Depending on the embodiment, the tubular body 105 may have a proximal segment 175 and/or a distal segment 180 that is formed of portions that successively decrease in durometer moving distally along the tubular body 105. For example, the proximal segment 175 could be formed of a most proximal portion and several intermediate proximal portions between the most proximal portion and the distal segment 180. Similarly, the distal segment 180 could be formed of a most distal portion and several intermediate distal portions between the most distal portion and the proximal segment 175. In such an embodiment, the tubular body 105 may be the most rigid and stiff at the most proximal portion of the proximal segment 175 and become progressively softer (i.e., less rigid and stiff) over each intermediate proximal portion and intermediate distal portion moving distally towards the most distal portion of the distal segment 180. Thus, in such an embodiment, the tubular body 105 gets progressively softer along its length moving proximal to distal along the length of the tubular body 105. For example, in one embodiment, the most proximal portion of the proximal segment 175 is formed of 72D PEBAX®. Moving distally along the tubular body 105 from the most proximal portion of the proximal segment 175, the tubular body 105 transitions into 63D PEBAX® for the intermediate proximal portion of the proximal segment 175 immediately distally adjacent the most proximal portion of the proximal segment 175. Continuing distally along the tubular body 105, the tubular body again transitions to 55D PEBAX® for the next intermediate proximal portion of the proximal segment 175. Continuing distally along the tubular body 105, each intermediate proximal portion of the proximal segment 175 and each intermediate distal portion of the proximal segment 180 successively decreases in durometer as compared to its immediately proximal neighbor intermediate proximal portion or intermediate distal portion.

The lumens 165, 170 may be formed of a lubricious material such as, for example, polytetrafluoroethylene (“PTFE®”), thereby facilitating the lead 65 and skeleton legs 200 sliding along the respective lumens 165, 170. Construction methods for the tubular body 105 and its proximal section 175 and distal section 180 may include various extrusion, molding and/or reflow processes as known in the art for manufacturing tubular bodies of catheters and sheaths. The tubular body 105 may be configured to be slittable or peelable via methods and configurations know in the art.

As indicated in FIGS. 4 and 6, the longitudinally extending skeleton 110 may include a proximal end 185, a distal end 190, a hub 195 forming the proximal end 185, and one or more legs 200 extending distally from the hub 195 to form a longitudinally extending portion 205 of the skeleton 110 and the distal end 190 of the skeleton 110. A skeleton leg 200 may take the form of a flat wire or round wire or have a shaped structural transverse cross section such as, for example, a C-beam, I-beam or L-beam transverse cross section that optimizes the mechanical performance of the skeleton leg 200 with respect to moment of inertia. A skeleton 110 or its legs 200 may employ a combination of cross sections. For example, one part of a skeleton leg may be a flat wire and another part of a skeleton leg may have C-beam configuration, and the other skeleton leg may have be a round wire along its entire length. The intra-wall lumens 170 may have transverse cross sections that generally match the transverse cross sections of the corresponding skeleton legs 200.

Each leg 200 includes a distal end 210 and a proximal end 215. The leg proximal ends 215 are coupled together in a fixed spaced-apart arrangement by the skeleton hub 195. More specifically, the leg proximal ends 215 may be imbedded in the skeleton hub 195 via molding or other processes or methods known in the art. The fixed spaced-apart arrangement generally of the legs 200 of the skeleton 110 matches the fixed spaced-apart arrangement of the intra-wall lumens 170 of the tubular body 105. The leg distal ends 210 are free of connection to each other. As a result of the proximal legs ends 215 being in a fixed spaced-apart arrangement matching the spaced-apart arrangement of the intra-wall lumens 170 of the tubular body 105 and the leg distal ends 210 being free of connection to each other, the leg distal ends 210 can be positioned relative to the proximal openings 217 of the respective intra-wall lumens 170 such that the each leg 200 can be fed distally through its respective intra-wall lumen 170 as the skeleton 110 is received in the tubular body 105 to transition the catheter 100 from the disassembled state depicted in FIG. 6 to the assembled state depicted in FIG. 4.

In one embodiment, the skeleton legs 200 are in a spaced-apart arrangement wherein the skeleton legs 200 are evenly radially spaced-apart from each other. For example, in one embodiment, skeleton legs 200 are on opposite sides of the skeleton 110 (i.e., spaced radially apart by 180 degrees).

The hub 195 of the skeleton 110 may be formed of a polymer material such as, for example, PEBAX®, ABS, Nylon, Polyethelene, or etc. The proximal ends 215 of the skeleton legs 200 may be received within the hub 195 of the skeleton 110 and fixedly secured thereto via an adhesive, molding, or other methods known in the art.

As shown in FIGS. 4 and 6, the hub 195 of the skeleton 110 includes an outer circumferential surface 230 and an inner circumferential surface 235. The inner circumferential surface 235 of the skeleton hub 195 defines a segment of the central or lead delivery lumen 165. When the skeleton 110 is received in the tubular body 105, the segment of the central lumen 165 formed by the tubular body 105 and the tubular body hub 125 forms the complete central lumen 165 of the catheter 100. In other words, the hubs 125, 195 each have central holes that align with each other and a longitudinal axis of the tubular body when the skeleton legs are received in the respective intra-wall lumens.

A second mechanical engagement feature 240 is located on the outer circumferential surface 230 of the skeleton hub 195 and configured to mechanically engage the first mechanical engagement feature 130 on the tubular body hub 125. For example, in one embodiment, the first engagement feature 130 may be one or more lips, bumps or tabs 130 that are mechanically engaged by the second mechanical engagement feature 240, which may be one or more biased lever arms 240. In other embodiments, the location of the lips, bumps or tabs and the lever arms may be reversed such the lips, bumps or tabs are on the hub 195 of the skeleton 110, and the lever arms are located on the hub 125 of the tubular body 105. In yet other embodiments, the first and second mechanical engagement features 130, 240 may combine to form mechanical engagement arrangements such as, for example, a bayonet lock, a luer lock, a latch or other such mechanical engagement arrangement as known in the art.

In one embodiment, either or both of the skeleton hub 195 and the tubular body hub 125 may be configured to also serve as a hemostasis valve. In one embodiment, the skeleton hub 195 may be configured to allow an additional hemostasis valve to be mounted on the proximal end of the skeleton hub 195.

In one embodiment, as illustrated in FIGS. 4 and 6, each skeleton leg 200 may include a proximal segment 220 and a distal segment 225. The proximal segment may 220 may form the majority of the length of the skeleton longitudinal portion 205, the distal segment 225 forming the remainder of the length of the skeleton longitudinal portion 205. The proximal segment 220 and distal segment 225 may have different constructions and/or treatments and/or different stiffness and/or torqueability. In one embodiment, for example, the proximal segment 220 may be heat treated to a different extent than the distal segment 225.

As shown in FIG. 6, in one embodiment, the proximal segment 220 may be generally straight, while the distal segment 225 has a predetermined or desired curve or non-linear shape such that the distal segment 225 self-biases to assume the curve non-linear shape when not acted on by another force. The legs 200 may be formed of a super-elastic material, such as, for example, Nickel-Titanium alloys (e.g., Nitinol), copper-zinc-aluminum alloy, copper-aluminum-nickel alloy, iron-manganese-silicon alloy, or etc. or other material able to maintain the desired curve or non-linear shape.

At least in part because of the material and configuration of some embodiments of the legs 200 employed by the catheter 100, the catheter is less likely to suffer from inadvertent loss of its preferred shape during lead implantation, as compared to catheters known in the art. Specifically, the catheter 100 is able to maintain its curved shape longer with a lesser degree of change in curve angle over the course of a lengthy procedure. This ability to maintain its designed curvature is at least in part due to the catheter 100 employing, in some embodiments, a completely metal skeleton 110, which is less likely to soften and relax, as compared to known heat treated polymer-metal composite structures, when the catheter is placed in the wet environment of the venous vasculature.

As can be understood from FIGS. 4 and 5, when a shaped catheter 100 is desired during the implantation of a lead 65, the skeleton 110 being received in the tubular body 105 of the catheter 100 (i.e., the catheter 100 is in the assembled state depicted in FIGS. 4 and 5) causes the catheter 100 and, more specifically, the tubular body distal segment 180, to generally assume the curved configuration of the skeleton distal segment 225. Thus, the catheter 100, when in the assembled state depicted in FIG. 4 has the shape and rigidity to allow the catheter distal end 115 to be navigated as desired through the patient's vasculature and cardiac anatomy. The catheter 100 may be maintained in the assembled state depicted in FIGS. 4 and 5 via the mechanical engagement of the first and second mechanical engagement features 130, 240 discussed above.

As can be understood from FIG. 6, when the skeleton 110 is removed from the tubular body 105 (i.e., the catheter 100 is in the disassembled state depicted in FIG. 6), the tubular body 105 and, more specifically, the tubular body distal segment 180, is free to assume its natural state (e.g., a straight or even highly flexible or floppy condition). Accordingly, the likelihood or magnitude of whip at the tubular body distal end 115 is substantially reduced when the tubular body 105 is proximally withdrawn from about an implanted medical lead 65. The catheter 100 may be disassembled from the assembled state depicted in FIGS. 4 and 5 to the disassembled state depicted in FIG. 6 via disengaging the mechanical engagement of the first and second mechanical engagement features 130, 240 and withdrawing the skeleton 110 from within the tubular body 105 and, more specifically, the skeleton legs 200 from within the intra-wall lumens 170. Thus, decoupling the skeleton hub 195 from the tubular body hub 125 and proximally displacement of the skeleton hub 195 relative to the tubular body hub 125 causes the skeleton legs 200 to withdraw from the respective intra-wall lumens 170. As a result, the tubular body distal segment 180 is free to assume its natural, non-externally biased shape, which may be generally linear or less curved than the non-linear shape the skeleton 110 caused the tubular body distal segment 180 to assume when the catheter 100 was in the assembled state.

In one embodiment, upon withdrawal of the skeleton legs 200 from the intra-wall lumens 170, the tubular body distal segment 180 by itself is less rigid than a combined rigidity of the tubular body distal segment 180 and the corresponding distal portion 225 of the skeleton legs 200.

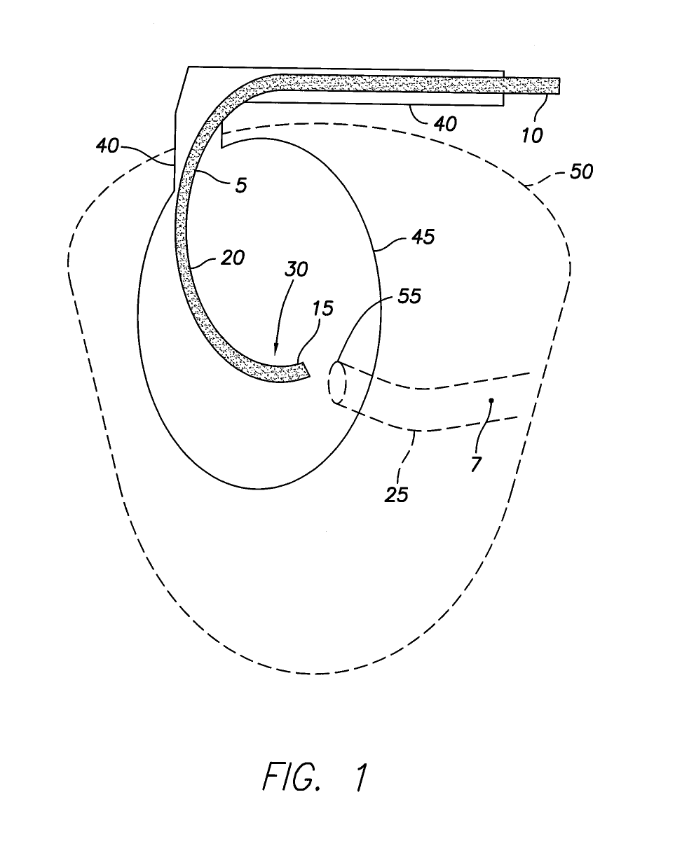

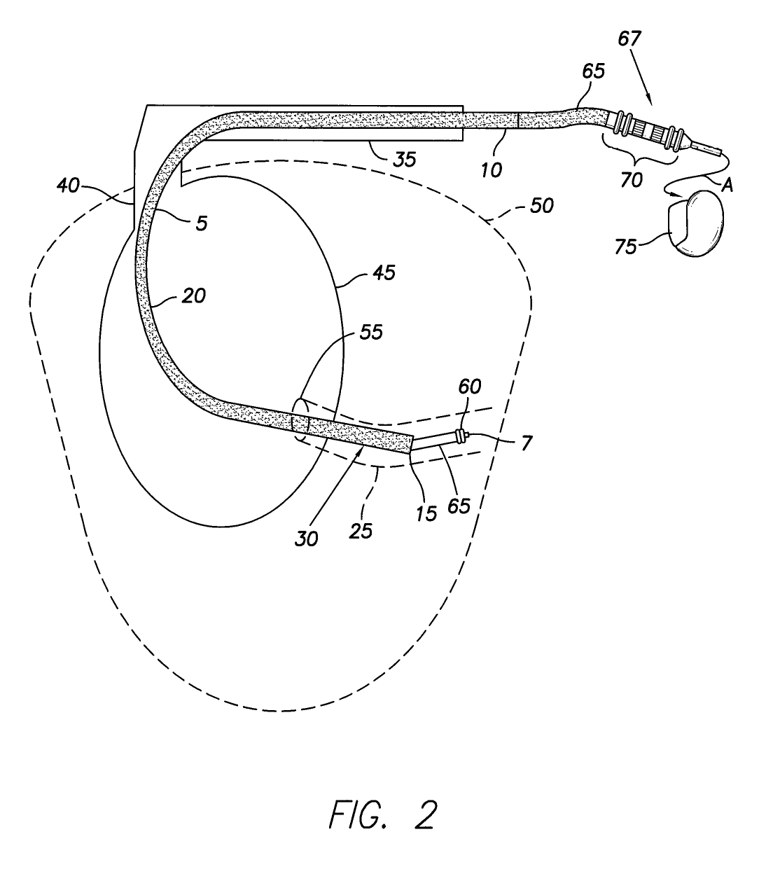

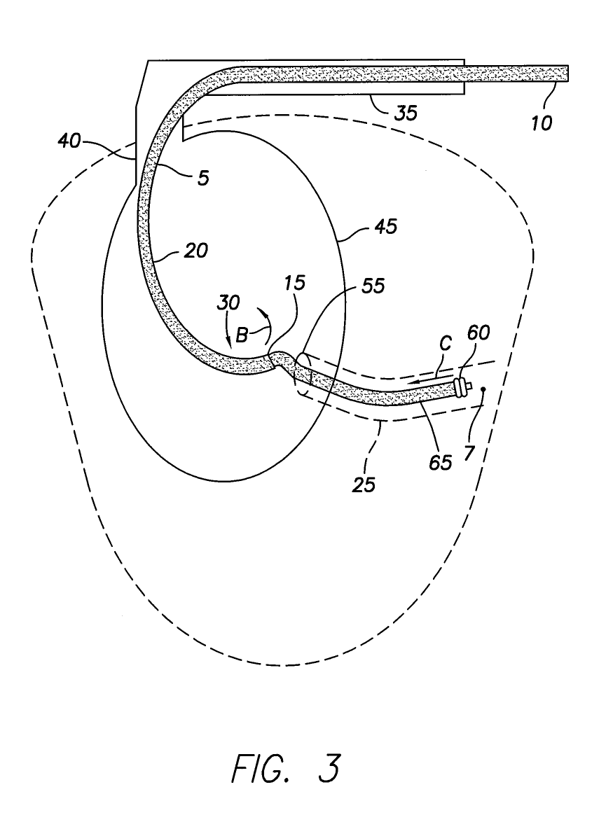

In one embodiment, the tubular body proximal portion 175 is substantially more stiff and/or torqueable than the tubular body distal portion 180. For example, in one embodiment, the rigidity of the tubular body proximal portion 175 may be such that the combined rigidity of the tubular body proximal portion 175 and the skeleton proximal portion 220 when the catheter 100 is in the assembled state is not substantially greater than the rigidity of the tubular body proximal portion 175 by itself when the catheter 100 is in the disassembled state. However, in such an embodiment, the rigidity of the tubular body distal portion 180 may be such that the combined rigidity of the tubular body distal portion 180 and the skeleton distal portion 225 when the catheter 100 is in the assembled state is substantially greater than the rigidity of the tubular body distal portion 180 by itself when the catheter 100 is in the disassembled state. Also, in such an embodiment, while the skeleton leg distal portions 225 may be sufficiently rigid to cause the tubular body distal portion 180 to deflect into a curved configuration when the catheter 100 is in the assembled state, the skeleton leg distal portions 225 are not so rigid as to be able to cause the tubular body proximal portion 175 to deflect to any significant extent into a curved configuration when the skeleton leg distal portions 225 are proximally displacing through the intra-wall lumens 170 of the tubular body proximal portion 175 when the catheter 100 is transitioning into the disassembled state. Ultimately, in such an embodiment, material selection and braid patterns for the proximal and distal tubular body sections 175, 180 are selected to allow for a soft and pliable tubular body distal section 180 that is readily controlled in shape and stiffness via the skeleton leg distal section 225 and a tubular body proximal section 175 that is not significantly impacted with respect to shape or stiffness via the skeleton leg distal section 180. As a result, once the lead distal end 60 is located via the assembled catheter 100 (i.e., as represented in FIGS. 4 and 5) at a desired implantation site within or on the patient's heart, the catheter 100 can be transitioned to the disassembled state (i.e., as represented in FIG. 6) and in making the transition, the likelihood or magnitude of whip at the tubular body distal end 115 is substantially reduced as compared to catheters similar to those described above with respect to FIGS. 1-3.

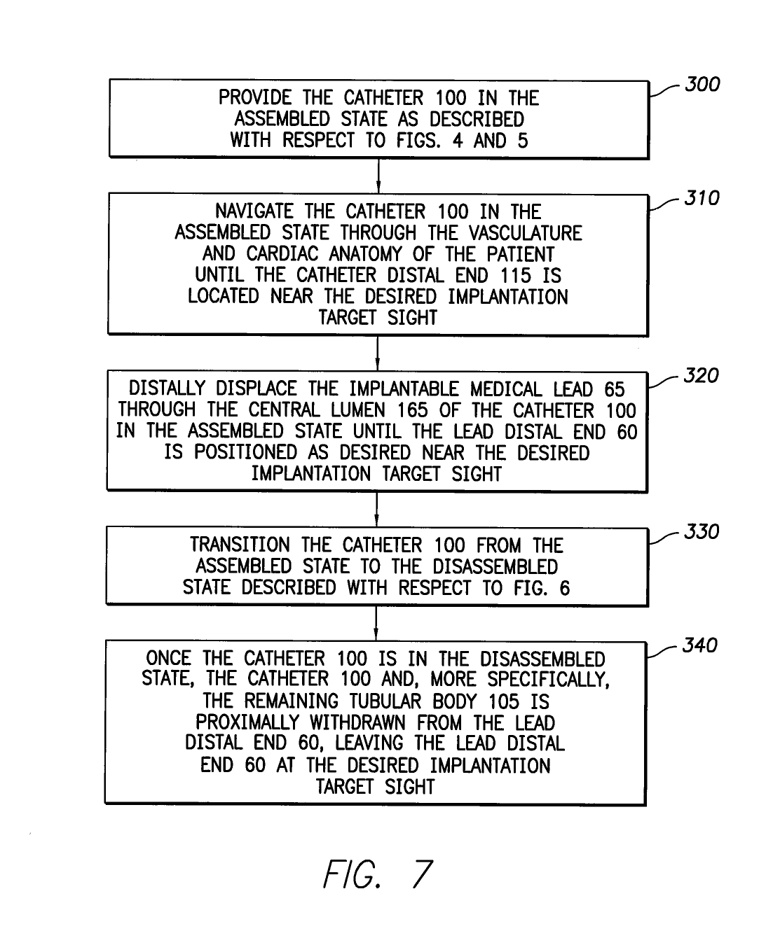

For a discussion of a method of employing the catheter 100 to deliver a distal end 60 of an implantable medical lead 65 to a desired implantation target site while employing the catheter's features to minimize the potential for whip, reference is now made to FIG. 7, which is a flow chart outlining the method. The delivery method may begin by providing the catheter 100 in the assembled state described above with respect to FIGS. 4 and 5 [block 300 of FIG. 7]. The catheter 100 in the assembled state is navigated through the vasculature and cardiac anatomy of the patient until the catheter distal end 115 is located near the desired implantation target site [block 310 of FIG. 7]. The implantable medical lead 65 is distally displaced through the central lumen 165 of the catheter 100 in the assembled state until the lead distal end 60 is positioned as desired near the desire implantation target site [block 320 of FIG. 7]. The catheter 100 is then transitioned from the assembled state to the disassembled state described above with respect to FIG. 6 [block 330 of FIG. 7]. More specifically, in one embodiment, transitioning the catheter 100 from the assembled state to the disassembled state includes maintaining the position of the tubular body distal end 115 near the desired implantation target site as the skeleton 110 is decoupled and removed from the tubular body 105. Once the catheter 100 is in the disassembled state, the catheter 100 and, more specifically, the remaining tubular body 105 is proximally withdrawn from the lead distal end 60, leaving the lead distal end 60 at the desired implantation target site [block 340 of FIG. 7]. The tubular body 105 may then be slit or peeled as needed to allow it to be removed completely from the implanted medical lead 65.

In the context of employing the catheter 100 to implant a lead 65 in a confined space such as, for example, the coronary sinus (or, more specifically, a sub branch of the coronary sinus), in one embodiment, the catheter 100 is positioned in the assembled state in the coronary sinus and sub branch thereof such that the at least a distal part of the tubular body proximal portion 175 extends into the coronary sinus and sub branch thereof (i.e., the confining space). Upon placement of the lead distal end 60 at the implantation target site, the catheter 100 is transitioned from the assembled state to the disassembled state while maintaining the position of the tubular body distal end 115 near the desired implantation target site as the skeleton 110 is decoupled and removed from the tubular body 105. Thus, in the context of the implantation of a LV lead in the coronary sinus and sub branch thereof, the confines of the coronary sinus and sub branch thereof may be employed to prevent displacement of the tubular body distal portion 180, which is readily deflectable by the skeleton 110 absent the confines of the coronary sinus and sub branch thereof, as the skeleton leg distal portions 225 are proximally withdrawn along the lumens 170 into the tubular body proximal portion 175, which at least partially extends into the coronary sinus and is not deflectable to any significant extent by the passage of the skeleton leg distal portions 225 through the tubular body proximal portion 175. As the tubular body distal portion 180 is constrained from whip by the confines of the surrounding coronary sinus and the tubular body proximal portion 175 is constrained by the coronary sinus at the distal end of the tubular body proximal portion 175 and further the tubular body proximal portion 175 is not deflectable by the passage of the skeleton leg distal portions 225, the potential for catheter whip is effectively eliminated.

In one embodiment of employing the catheter 100 to implant a lead 65 in a confined space such as, for example, the coronary sinus (or, more specifically, a sub branch of the coronary sinus), the catheter 100 may be transitioned between the assembled state and disassembled state to modify the deflection of the catheter distal end 115 as needed to facilitate the negotiation of the distal end 115 through the vasculature of the patient to the lead implantation site. For example, the skeleton 110 and tubular body 105 can be decoupled from each other at their respective proximal ends and the skeleton 110 and, as needed, the skeleton 110 can be withdrawn, to a greater or lesser extent, from the tubular body 105 and reinserted, to a greater or lesser extent, into the tubular body 105 to modify the curvature and deflectability of the distal end 115 to facilitate the distal end 115 being negotiated through the patient vasculature to the implantation site. Thus, although the skeleton 110 is part of the catheter 100, by modifying the extent to which the skeleton 110 is received in the tubular body 105, the catheter 100 can be operated as a deflectable catheter and steered towards the implant location.

By partially withdrawing the skeleton 110 from the tubular body 105, the distal end 115 can be partially relaxed, thereby acting as a deflectable catheter during coronary sinus branch sub-selection. If lead placement is deemed inappropriate (for any reason such as insufficient pacing response, etc.) during the implant procedure and prior to removing the catheter completely, the skeleton 110 can again be partially withdrawn from the tubular body to ease the catheter curve gradually at the distal end 115, providing a range of possible curve shapes that may facilitate the lead redeployment to a new location. Thus the catheter 100 can operate as a deflectable catheter based on the degree to which the skeleton 110 is disengaged and pulled back from the hub of the tubular body.

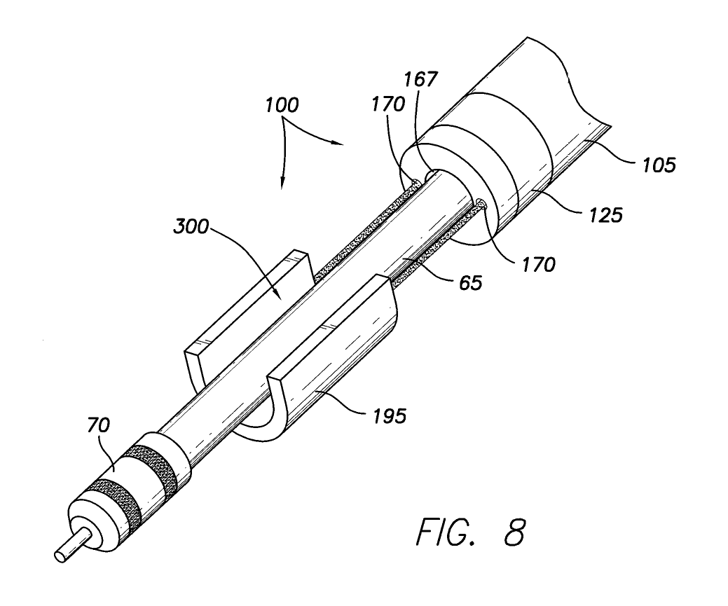

As shown in FIG. 8, which is an isometric view of the proximal ends of the skeleton 110 and tubular body 105 with a lead 65 extending into the catheter 100, in one embodiment, the skeleton hub 195 has a U-shaped or open channel configuration. Specifically, the hub 195 has an open side 300 that extends the length of the hub 195 so as to allow the lead connector end 70 to clear the hub 195 when the catheter 100 is being removed from about the lead 65 once the lead has been implanted.

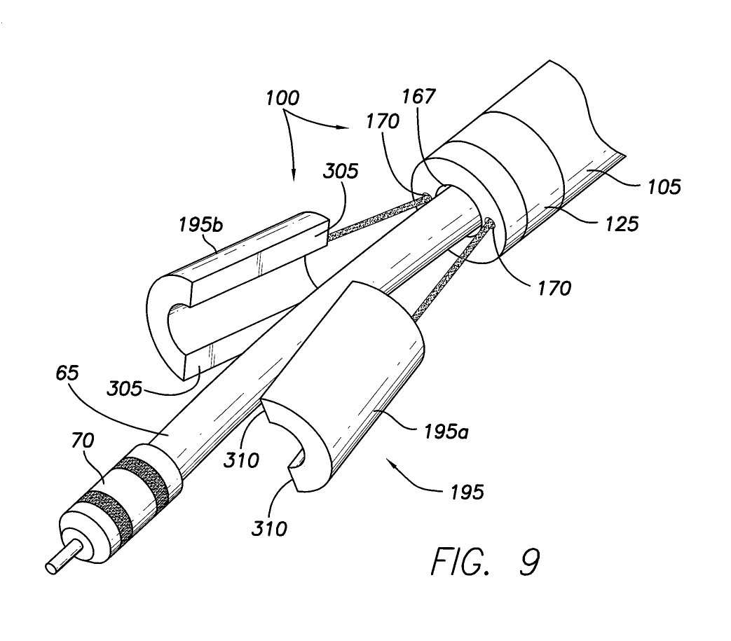

As illustrated in FIG. 9, which is an isometric view of the proximal ends of the skeleton 110 and tubular body 105 with a lead 65 extending into the catheter 100, in one embodiment, the skeleton hub 195 has splittable configuration. Specifically, the hub 195 has a first portion 195a and a second portion 195b. The portions 195a, 195b are coupled together when the longitudinally extending sides 305, 310 of each portion 195a, 195b are interfaced together such that the coupled-together portions 195a, 195b form an integrated or unitary hub structure 195. The portions 195a, 195b may be maintained in the integrated or unitary hub arrangement via a mechanical engagement feature (e.g., a member that extends across the interfaced sides 305, 310, sides 305, 310 that are slotted to engage with each other in a longitudinal sliding arrangement or sides 305, 310 that form an interference or friction fit with each other). The portions 195a, 195b may be maintained in the integrated or unitary hub arrangement via stress riser features (e.g., a V-groove, skive, etc.) extending along the longitudinal length of the hub 195 and forming the interfaced sides 305, 310 when the portions are splittable (e.g., split) from each other. The hub 195 may be split or separated into portions 195a, 195b to allow the lead connector end 70 to clear the hub 195 when the catheter 100 is being removed from about the lead 65 once the lead has been implanted.

Although the present invention has been described with reference to preferred embodiments, persons skilled in the art will recognize that changes may be made in form and detail without departing from the spirit and scope of the invention.

What is claimed is:

1. A delivery tool for the delivery of an implantable medical lead, the tool comprising:
a longitudinally extending tubular body including a distal end, a proximal end, a tubular body segment proximal the distal end, a first lumen extending between the proximal and distal ends, and a second lumen, wherein the first lumen is configured to receive therein the implantable medical lead; and
a longitudinally extending skeleton received in the second lumen and including a distal end, a proximal end, and a portion near the distal end that biases into a non-linear shape, wherein the portion of the skeleton causes the tubular body segment to generally assume the non-linear shape and the skeleton is withdrawable from the second lumen.

2. The tool of claim 1, wherein the first lumen includes a central lumen of the tubular body.

3. The tool of claim 1, wherein the second lumen includes a wall lumen.

4. The tool of claim 1, wherein the second lumen includes a first wall lumen and a second wall lumen radially separated from the first wall lumen, and the skeleton further includes a first leg received in the first wall lumen and a second leg proximally coupled to the first leg and received in the second wall lumen.

5. The tool of claim 4, wherein the first and second wall lumens are radially separated by approximately 180 degrees.

6. The tool of claim 4, wherein the second leg is proximally coupled to the first leg via a structural element of the proximal end of the skeleton, wherein proximal displacement of the structural element relative to the proximal end of the tubular body causes the first and second legs to respectively withdraw from the first and second wall lumens.

7. The tool of claim 6, wherein the structural element includes a first hub.

8. The tool of claim 7, wherein the first hub includes a U-shaped cross section.

9. The tool of claim 7, wherein the first hub includes a first portion and second portion splittable from the first portion.

10. The tool of claim 9, wherein the first hub includes a stress concentration line or a mechanical coupling arrangement that allows the second portion to be splittable from the first portion.

11. The tool of claim 7, wherein the proximal end of the tubular body includes a second hub, the first and second hubs each having central holes that align with each other and a longitudinal axis of the tubular body when the first and second legs are respectively received in the first and second wall lumens.

12. The tool of claim 11, wherein the first and second hubs have a mechanical engagement arrangement that mechanically engages the hubs together in a manner that may be released to allow the hubs to be separated from each other.

13. The tool of claim 12, wherein the mechanical engagement arrangement includes a bayonet lock, a luer lock, a biased lever arm, or a latch.

14. The tool of claim 1, wherein proximal ends of the skeleton and the tubular body have a mechanical engagement arrangement that mechanically engages the proximal ends together in a manner that may be released to allow the proximal ends to be separated from each other.

15. The tool of claim 14, wherein the mechanical engagement arrangement includes a bayonet lock, a luer lock, a biased lever arm, or a latch.

16. The tool of claim 1, wherein the tubular body is slittable or peelable.

17. The tool of claim 1, wherein the skeleton includes an I-beam or C-beam cross section.

18. The tool of claim 1, wherein the skeleton includes a super-elastic metal.

19. The tool of claim 1, wherein the skeleton includes a leg received in the second lumen, and the leg is formed completely of metal.

20. The tool of claim 1, further comprising a hemostatic valve mounted on the proximal end of the tubular body.

21. The tool of claim 1, wherein, upon withdrawal of the skeleton from the second lumen, the tubular body segment is free to assume a shape different from the non-linear shape.

22. The tool of claim 21, wherein the non-linear shape is curved.

23. The tool of claim 22, wherein the shape different from the non-linear shape is at least one of generally linear or less curved than the non-linear shape.

24. The tool of claim 22, wherein, upon withdrawal of the skeleton from the second lumen, the tubular body segment by itself is less rigid than a combined rigidity of the tubular body segment and the portion of the skeleton.

25. A method of delivering a distal end of an implantable medical lead to a target implant site in a patient, the method comprising:
(a) providing a delivery tool in an assembled state, wherein the delivery tool in the assembled state includes: a longitudinally extending tubular body including a distal end, a proximal end, a tubular body segment proximal the distal end, a first lumen extending between the proximal and distal ends, and a second lumen; and a longitudinally extending skeleton received in the second lumen and including a distal end, a proximal end, and a portion near the distal end that biases into a non-linear shape, wherein the portion of the skeleton causes the tubular body segment to generally assume the non-linear shape;
(b) tracking the delivery tool in the assembled state through a route in the patient;
(c) distally displacing the implantable medical lead through the first lumen of the delivery tool in the assembled state until the a distal end of the implantable medical lead is located as desired near the target implant site;
(d) upon achieving step (c), transitioning the delivery tool from the assembled state to a disassembled state, wherein the delivery tool in the disassembled state has the skeleton removed from the second lumen; and
(e) upon achieving step (d), proximally withdrawing the distal end of the tubular body from the distal end of the implantable medical lead.

26. The method of claim 25, wherein transitioning the delivery tool from the assembled state to the disassembled state includes maintaining a position of the distal end of the tubular body relative to the target implant site as the skeleton is being proximally displaced within the second lumen.

27. The method of claim 26, wherein the tubular body includes a proximal portion proximal the tubular body segment and more rigid than the tubular body segment, wherein transitioning the delivery tool from the assembled state to the disassembled state includes maintaining a most distal part of the proximal portion within a confining region of the patient as the delivery tool is transitioned from the assembled state to the disassembled state.
